(19) United States
(12) Patent Application Publication
(10) Pub. No.: US 2009/0111742 A1
(43) Pub. Date: Apr. 30, 2009

(54) Use of fgf-21 and thiazolidinedione for treating type 2 diabetes

(76) Inventors: Alexei Kharitonenkov, Zionsville, IN (US); Tatiyana Leonidovna Shiyanova, Carmel, IN (US)

Correspondence Address: ELI LILLY & COMPANY, PATENT DIVISION, P.O. BOX 6288, INDIANAPOLIS, IN, 46206-6288, US

(21) Appl. No.: 10/587,138

(22) PCT Filed: Jan. 19, 2005
(86) PCT No.: PCT/US05/00023

§ 371 Date: Jul. 21, 2006

Related U.S. Application Data
(60) Provisional application No. 60/539,241, filed on Jan. 26, 2004.

(51) Int. Cl. A61K 38/18; A61K 31/443

(52) U.S. Cl. 514/12; 514/340

(57) ABSTRACT

A method for treating type 2 diabetes and metabolic syndrome comprising administering an effective amount of fibroblast growth factor 21 in combination with a thiazolidinedione.

### FIELD OF INVENTION

This invention relates to the use of fibroblast growth factor 21 in combination with a thiazolidinedione for the treatment of mammals suffering from non-insulin dependent Diabetes Mellitus (NIDDM: Type 2).

### DESCRIPTION OF THE ART

Type 2 diabetes is a debilitating disease characterized by high-circulating blood glucose, insulin and corticosteroid levels. The incidence of type 2 diabetes is high and rising and is becoming a leading cause of mortality, morbidity and healthcare expenditure throughout the world (Amos et al., Diabetic Med. 14:S1-85, 1997). The causes of type 2 diabetes are not well understood. It is thought that both resistance of target tissues to the action of insulin and decreased insulin secretion (“β-cell failure”) occur. Major insulin-responsive tissues for glucose homeostasis are liver, in which insulin stimulates glycogen synthesis and inhibits gluconeogenesis; muscle, in which insulin stimulates glucose uptake and glycogen and inhibits lipolysis. Thus, as a consequence of the diabetic condition, there are elevated levels of glucose in the blood, and prolonged high blood sugar that is indicative of a condition which will cause blood vessel and nerve damage.

Currently, there are various pharmacological approaches for the treatment of type 2 diabetes (Scheen et al., Diabetes Care, 22(9):1568-1577, 1999). One such approach is the use of thiazolidinediones (TZDs), which represent a new class of oral antidiabetic drugs that improve metabolic control in patients with type 2 diabetes. Their glucose-lowering effect is mediated through the improvement of insulin sensitivity. They reduce insulin resistance in adipose tissue, muscle and liver (Oakes et al., Metabolism 46:935-942, (1997); Young et al. Diabetes 44:1087-1092, (1995); Oakes et al., Diabetes 43:1203-1210, (1994); Smith et al., Diabetes Obes Metab 2:363-372 (2000)). In addition, free fatty acid (FFA) levels were lowered and there was a marked reduction in triglycerides.

Fibroblast growth factor 21 (FGF-21) belongs to a family of large polypeptides widely expressed in developing and adult tissues (Baird et al., Cancer Cells, 3:239-243, 1991) that play crucial roles in multiple physiological functions including angiogenesis, mitogenesis, pattern formation, cellular differentiation, metabolic regulation and repair of tissue injury (McKeehan et al., Prog. Nucleic Acid Res. Mol. Biol. 59:135-176, 1998). According to the published literature, the FGF family now consists of at least twenty-three members, FGF-1 to FGF-23 (Reuss et al., Cell Tissue Res. 313:139-157 (2003).

FGF-21 has been reported to be preferentially expressed in the liver (Nishimura et al., Biochimica et Biophysica Acta, 1492:203-206, (2000); WO01/36640; and WO01/18172) and recently, has been shown to stimulate glucose-uptake in mouse 3T3-L1 adipocytes after prolonged treatment, in the presence and absence of insulin, and to decrease fed and fasting blood glucose, triglycerides, and glucagon levels in ob/ob and db/db mice in a dose-dependant manner, thus, providing the basis for the use of FGF-21 as a therapy for treating diabetes and obesity (WO03/011213).

There is now rapidly growing evidence from clinical studies that TZDs administered alone or in combination with metformin have glucose-lowering effects in patients with type 2 diabetes combined with the ability to induce a reduction in plasma insulin concentrations (i.e. in hyperinsulinaemia) [Aronoff et al., Diabetes Care 2000; 23: 1605-1611]; Lebovitz et al., J Clin Endocrinol Metab 2001; 86: 280-288; Phillips et al. Diabetes Care 2001; 24: 308-315]. In addition, other parameters of the metabolic syndrome are also significantly improved, including lipid disturbances [Day C. Diabet Med 1999; 16: 179-192; Ogihara et al. Am J Hypertens 1995; 8: 316-320], high blood pressure [Ogihara et al. Am J Hypertens 1995; 8: 316-320] and impaired fibrinolysis [Gottschling-et al. Diabetologia 2000; 43:.377-383]. However, there are numerous side effects associated with the use of TZDs such as weight gain, liver toxicity, upper respiratory tract infection, headache, back pain, hyperglycemia, fatigue, sinusitis, diarrhea, hypoglycemia, mild to moderate edema, and anemia (Moller, D., Nature, 2001, 414: 821-827).

Accordingly, there is a need for an improved therapy of type 2 diabetes that has fewer adverse effects than the available pharmaceutical approaches utilizing TZDs. The present invention provides a combination therapy of FGF-21 with a TZD resulting in a synergistic effect that enhances insulin sensitivity in peripheral tissues, stimulates glucose uptake and has fewer adverse effects than treatment regimens for type 2 diabetes using TZDs alone or in combination with other agents.

### SUMMARY OF THE INVENTION

The present invention provides a method for treating a mammal exhibiting type 2 diabetes or metabolic syndrome comprising: administering to said mammal a therapeutically effective amount of FGF-21 or an FGF-21 compound in combination with a thiazolidinedione sufficient to achieve in said mammal at least one of the following modifications: reduction in triglycerides, decrease in insulin resistance, reduction of hyperinsulinemia, increase in glucose tolerance, or reduction of hyperglycemia.

### DETAILED DESCRIPTION OF THE INVENTION

FGF-21 is a 208 amino acid polypeptide containing a 27 amino acid leader sequence. Human FGF-21 is highly identical to mouse FGF-21 (˜79% amino acid identity) and rat FGF-21 (˜80% amino acid identity). Human FGF-21 is the preferred polypeptide of the present invention but it is recognized that one with skill in the art could readily use analogs, muteins, or derivatives of human FGF-21 or an alternative mammalian FGF-21 polypeptide sequence for the uses described herein.

The mature human 181 amino acid FGF-21 polypeptide is shown below (SEQ ID NO:1):

```
1 10 20
His Pro Ile Pro Asp Ser Ser Pro Leu Leu Gln Phe Gly Gly Gln Val Arg Gln Arg Tyr
                                                                        30 40
Leu Tyr Thr Asp Asp Ala Gln Gln Thr Glu Ala His Leu Glu Ile Arg Glu Asp Gly Thr
                                                                        50 60
Val Gly Gly Ala Ala Asp Gln Ser Pro Glu Ser Leu Leu Gln Leu Lys Ala Leu Lys Pro
                                                                        70 80
Gly Val Ile Gln Ile Leu Gly Val Lys Thr Ser Arg Phe Leu Cys Gln Arg Pro Asp Gly
                                                                        90 100
Ala Leu Tyr Gly Ser Leu His Phe Asp Pro Glu Ala Cys Ser Phe Arg Glu Leu Leu Leu
                                                                        110 120
Glu Asp Gly Tyr Asn Val Tyr Gln Ser Glu Ala His Gly Leu Pro Leu His Leu Pro Gly
                                                                        130 140
Asn Lys Ser Pro His Arg Asp Pro Ala Pro Arg Gly Pro Ala Arg Phe Leu Pro Leu Pro
                                                                        150 160
Gly Leu Pro Pro Ala Leu Pro Glu Pro Pro Gly Ile Leu Ala Pro Gln Pro Pro Asp Val
                                                                        170 180
Gly Ser Ser Asp Pro Leu Ser Met Val Gly Pro Ser Gln Gly Arg Ser Pro Ser Tyr Ala
Ser
```

The corresponding DNA sequence coding for the mature human 181 amino acid FGF-21 polypeptide is (SEQ ID NO:2):

CACCCCATCCCTGACTCCAGTCCTCTCCTGCAATTCGGGGGCCAAGTCCG
GCAGCGGTACCTCTACACAGATGATGCCCAGCAGACAGAAGCCCACCTGG
AGATCAGGGAGGATGGGACGGTGGGGGGCGCTGCTGACCAGAGCCCCGAA
AGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATG
GATCGCTCCACTTTGACCCTGAGGCCTGCAGCTTCCGGGAGCTGCTTCTT
GAGGACGGATACAATGTTTACCAGTCCGAAGCCCACGGCCTCCCGCTGCA
CCTGCCAGGGAACAAGTCCCCACACCGGGACCCTGCACCCCGAGGACCAG
CTCGCTTCCTGCCACTACCAGGCCTGCCCCCCGCACTCCCGGAGCCACCC
GGAATCCTGGCCCCCCAGCCCCCCGATGTGGGCTCCTCGGACCCTCTGAG
CATGGTGGGACCTTCCCAGGGCCGAAGCCCCAGCTACGCTTCC

The FGF-21 useful in the methods of the present invention is preferably human FGF-21. Additionally, the methods of the present invention include the use of FGF-21 analogs, FGF-21 muteins, and FGF-21 derivatives hereinafter collectively known as FGF-21 compounds. FGF-21 compounds have sufficient homology to FGF-21 such that the compound has the ability to bind to the FGF-21 receptor and initiate a signal transduction pathway resulting in glucose uptake stimulation or other physiological effects as described herein. For example, FGF-21 compounds can be tested for glucose uptake activity using a cell-based assay such as that described in Example 1.

A human FGF-21 mutein is defined as comprising human FGF-21 in which at least one amino acid of the wild-type mature protein has been substituted by another amino acid. Examples of FGF-21 muteins are described in U.S. patent application 60/528,582 herein incorporated by reference. Generally speaking, a mutein possesses some modified property, structural or functional, of the wild-type protein. For example, the mutein may have enhanced or improved physical stability in concentrated solutions (e.g., less hydrophobic mediated aggregation), while maintaining a favorable bioactivity profile. The mutein may possess increased compatibility with pharmaceutical preservatives (e.g., m-cresol, phenol, benzyl alcohol), thus enabling the preparation of a preserved pharmaceutical formulation that maintains the physiochemical properties and biological activity of the protein during storage. Accordingly, muteins with enhanced pharmaceutical stability when compared to wild-type FGF-21, have improved physical stability in concentrated solutions under both physiological and preserved pharmaceutical formulation conditions, while maintaining biological potency. As used herein, these terms are not limiting, it being entirely possible that a given mutein has one or more modified properties of the wild-type protein.

An FGF-21 compound also includes a “FGF-21 derivative” which is defined as a molecule having the amino acid sequence of FGF-21 or an FGF-21 analog, but additionally having a chemical modification of one or more of its amino acid side groups, α-carbon atoms, terminal amino group, or terminal carboxylic acid group. A chemical modification includes, but is not limited to, adding chemical moieties, creating new bonds, and removing chemical moieties.

Modifications at amino acid side groups include, without limitation, acylation of lysine εamino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist. The α-carbon of an amino acid may be mono- or dimethylated.

Type 2 diabetes is characterized by excess glucose production in spite of the availability of insulin, and circulating glucose levels remain excessively high as a result of inadequate glucose clearance.

Glucose intolerance can be defined as an exceptional sensitivity to glucose.

Hyperglycemia is defined as an excess of sugar (glucose) in the blood.

Hypoglycemia, also called low blood sugar, occurs when your blood glucose level drops too low to provide enough energy for your body's activities.

Hyperinsulinemia is defined as a higher-than-normal level of insulin in the blood.

Insulin resistance is defined as a state in which a normal amount of insulin produces a subnormal biologic response.

Metabolic syndrome can be defined as a cluster of at least three of the following signs: abdominal fat—in most men, a 40-inch waist or greater; high blood sugar—at least 110 milligrams per deciliter (mg/dl) after fasting; high triglycerides—at least 150 mg/dL in the bloodstream; low HDL—less than 40 mg/dl; and, blood pressure of 130/85 or higher.

The FGF-21 administered according to this invention may be generated and/or isolated by any means known in the art such as described in Sambrook et al., Molecular Cloning: A Laboratory Manual, Cold Spring Harbor Laboratory Press, NY (1989).

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used for FGF-21.

TZDs are formulated as described in the art. For example, the TZDs rosiglitazone (Avandia™) and pioglitazone (Actos™) are currently used as pharmaceutical compositions administered alone or in combination with metformin or sulfonylureas for the treatment of type 2 diabetes.

The pharmaceutical compositions of the TZDs of the present invention may be administered by any means that achieve the generally intended purpose: to treat type 2 diabetes or metabolic syndrome. Preferably, the TZD is administered orally.

FGF-21 utilized in combination with a TZD may be formulated according to known methods to prepare pharmaceutically useful compositions. A desired formulation would be one that is a stable lyophilized product that is reconstituted with an appropriate diluent or an aqueous solution of high purity with optional pharmaceutically acceptable carriers, preservatives, excipients or stabilizers [Remington's Pharmaceutical Sciences 16th edition (1980)]. The FGF-21 of the present invention may be combined with a pharmaceutically acceptable buffer, and the pH adjusted to provide acceptable stability, and a pH acceptable for administration.

For parenteral administration FGF-21 is formulated generally, in a unit dosage injectable form (solution, suspension, or emulsion), with a pharmaceutically acceptable carrier, i.e. Preferably, one or more pharmaceutically acceptable anti-microbial agents may be added. Phenol, m-cresol, and benzyl alcohol are preferred pharmaceutically acceptable anti-microbial agents.

Optionally, one or more pharmaceutically acceptable salts may be added to adjust the ionic strength or tonicity. One or more excipients may be added to further adjust the isotonicity of the formulation. Glycerin, sodium chloride, and mannitol are examples of an isotonicity adjusting excipient.

“Pharmaceutically acceptable” means suitable for administration to a human. A pharmaceutically acceptable formulation does not contain toxic elements, undesirable contaminants or the like, and does not interfere with the activity of the active compounds therein.

If subcutaneous or an alternative type of administration is used, the FGF-21 compounds may be derivatized or formulated such that they have a protracted profile of action.

A “therapeutically effective amount” of FGF-21 or an FGF-21 compound is the quantity that results in a desired effect without causing unacceptable side-effects when administered to a subject. A desired effect can include an amelioration of symptoms associated with the disease or condition, a delay in the onset of symptoms associated with the disease or condition, and increased longevity compared with the absence of treatment. In particular, the desired effect is a reduction in blood glucose levels or triglerceride levels associated with type 2 diabetes or metabolic syndrome.

The phrase “in combination with” refers to the administration of FGF-21 with a TZD either simultaneously, sequentially or a combination thereof. Preferably, the TZD is administered orally and the FGF-21 is administered parenterally. The combination therapy of FGF-21 with a TZD results in a synergistic effect with enhanced efficacy in the treatment of type 2 diabetes. The synergy also results in a reduction of the dosage of the agents used in combination therapy resulting in reduced side effects such as weight gain, liver toxicity, upper respiratory tract infection, headache, back pain, hyperglycemia, fatigue, sinusitis, diarrhea, hypoglycemia, mild to moderate edema, and anemia.

The TZD utilized and the appropriate dose level is understood and appreciated in the art. A skilled artisan recognizes the appropriate dose level to use for each TZD to achieve a pharmaceutically effective amount for treating type 2 diabetes. TZDs agents suitable for use under the present invention include, but are not limited to, clinically recognized and commercially available agents such as rosiglitazone, pioglitazone, and troglitazone (Hauner, H., Diabetes Metab Res Rev 18:S10-S15 (2002)). Typically, the amount of rosiglitazone administered for the treatment of type 2 diabetes is from 4 mg to 8 mg per day and the amount of pioglitazone administered for the treatment of type 2 diabetes is from 15 mg to 45 mg per day.

The pharmaceutical compositions of the FGF-21 of the present invention may be administered by any means that achieve the generally intended purpose: to treat type 2 diabetes or metabolic syndrome. For example, administration may be by oral, ocular, optical, rectal, parenteral, intravaginal, topical (as by powders, ointments, drops, or transdermal patch), bucal, as an oral or nasal spray, or as ocular or intraotic drops. The term “parenteral” as used herein refers to modes of administration that include intravenous, intramuscular, intraperitoneal, intrastemal, subcutaneous, and intraarticular injection and infusion. The dosage administered will be dependent upon the age, health, and weight of the recipient, kind of concurrent treatment, if any, frequency of treatment, and the nature of the effect desired. Compositions within the scope of the invention include all compositions wherein FGF-21 is present in an amount that is effective to achieve the desired medical effect for treatment type 2 diabetes or metabolic syndrome. While individual needs may vary from one patient to another, the determination of the optimal ranges of effective amounts of all of the components is within the ability of the clinician of ordinary skill.

Those skilled in the art can readily optimize pharmaceutically effective dosages and administration regimens for therapeutic compositions comprising FGF-21, as determined by good medical practice and the clinical condition of the individual patient. A typical dose range for FGF-21 will range from about 0.01 mg per day to about 1000 mg per day for an adult. Preferably, the dosage ranges from about 0.1 mg per day to about 100 mg per day, more preferably from about 1.0 mg/day to about 10 mg/day. Most preferably, the dosage is about 1-5 mg/day. The appropriate dose of FGF-21 administered will result in lowering blood glucose levels and increasing energy expenditure by faster and more efficient glucose utilization, and thus is useful for treating type 2 diabetes or metabolic syndrome.

Alternatively, FGF-21 is administered twice weekly at a dose range from about 0.01 mg per dose to about 1000 mg per dose for an adult. Preferably, the dosage ranges from about 0.1 mg per dose to about 100 mg per dose, more preferably from about 1.0 mg per dose to about 10 mg per day. Most preferably, the dosage is about 1-5 mg per dose.

In another alternative, FGF-21 is administered once weekly at a dose range from about 0.01 mg per dose to about 1000 mg per dose for an adult. Preferably, the dosage ranges from about 0.1 mg per dose to about 100 mg per dose, more preferably from about 1.0 mg per dose to about 10 mg per dose. Most preferably, the dosage is about 1-5 mg per dose.

FGF-21 administered either daily, twice weekly or once weekly, combined with a TZD such as rosiglitazone or pioglitazone, has a synergistic effect in the treatment of type 2 diabetes that improves the efficacy of the TZD alone. Thus, this combination therapy reduces the therapeutic dose of the TZD required for therapeutic treatment of type 2 diabetes thereby minimizing the side effects typically observed with TZD therapy. For example the amount of TZD administered in combination with FGF-21 is reduced by about 10%, 20%, 30%, 40%, 50%, 60%, 70%, or to about 80% of the typical dose of TZD utilized in the treatment of type 2 diabetes.

In another aspect of the present invention, FGF-21 in combination with a TZD for use as a medicament for the treatment of type 2 diabetes or metabolic syndrome is contemplated.

Having now described the present invention in detail, the same will be more clearly understood by reference to the following examples, which are included herewith for purposes of illustration only and are not intended to be limiting of the invention.

All patents and publications referred to herein are expressly incorporated by reference.

### Preparation 1

### Expression and Purification of FGF-21 in E. coli

The bacterial expression vector pET30a is used for bacterial expression in this example. (Novagen, Inc., Madison, Wis.)). pET30a encodes kanamycin antibiotic resistance gene and contains a bacterial origin of replication (“ori”), a strong T7 phage-IPTG inducible promoter, a ribosome binding site (“RBS”), and suitable MCS with a number of unique restriction endonuclease cleavage sites. Conveniently for purification purpose, the vector can encode His- and S-tags for N-terminal peptide fusions, as well as, a C-terminal His-tag fusion. However, for purposes of the present invention, the cDNA encoding FGF-21 is inserted between restriction sites NdeI and BamHI, respectively, and the resulting construct does not take advantage of either of the described tags.

The nucleic acid sequence encoding FGF-2, lacking the leader sequence but substituted with a methionine residue, is amplified from a cDNA clone using PCR oligonucleotide primers, which anneal to the 5′ and 3′ ends of the open reading frame. Additional nucleotides, containing recognition sites for restriction enzymes NdeI and BamHI, are added to the 5′ and 3′ sequences, respectively.

For cloning, the 5′ forward and 3′ reverse PCR primers have nucleotides corresponding or complementary to a portion of the coding sequence of FGF-21-encoding nucleic acid according to methods known in the art. One of ordinary skill in the art would appreciate that the point in a polynucleotide sequence where primers begin can be varied.

The amplified nucleic acid fragments and the vector pET30a are digested with NdeI and BamHI restriction enzymes and the purified digested DNA fragments are then ligated together. Insertion of FGF-21 mutein-encoding DNA into the restricted pET30a vector places the FGF-21 mutein polypeptide coding region including its associated stop codon downstream from the IPTG-inducible promoter and in-frame with an initiating ATG codon. The associated stop codon, TAG, prevents translation of the six-histidine codons downstream of the insertion point,

The ligation mixture is transformed into competent E. coli cells using standard procedures such as those described in Current Protocols in Molecular Biology (John Wiley & Sons, Inc.).

Transformation reactions are plated on LB/Kanamycin plates and after an overnight growth transformants are picked for plasmid preparations or lysed in situ for screening by PCR. Positive recombinant plasmids, containing desired FGF-21 variant inserts, are identified by restriction analysis followed by DNA sequence analysis. Those plasmids are subsequently used to transform expression strains and protein production.

E. coli strains BL21(DE3), BL21(DE3)STAR or BL21(DE3) RP, are used for expressing FGF-21. These strains, which are only some of many that are suitable for expressing FGF-21, are available commercially from Novagen, Inc., Invitrogen and Stratagen, respectively. Transformants are identified by their ability to grow on LB plates in the presence of kanamycin.

Clones containing the desired constructs are grown overnight (o/n) in liquid culture in LB media supplemented with kanamycin (30 μg/ml). The o/n culture is used to inoculate a large culture, at a dilution of approximately 1:25 to 1:250. The cells are grown to an optical density of 0.6 (“OD600”) at 600 nm. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 12hours. Cells are then harvested by centrifugation, pellets washed with 50 mM Tris buffer, pH 8.0 and stored at −20° C. until purification. FGF-21 is expressed in the insoluble fraction i.e inclusion bodies (or granules) of E. coli. Although the expression level may a typically observed level for FGF-21 protein is 50 mg/L. The subsequent purification process starts with solubilization of the granules and refolding of the variants followed by four chromatographic steps.

To purify FGF-21 from E coli, the granules are solubilized in 50 mM Tris, pH 9.0, 7M Urea and 1 mM DTT through a pH ramp to pH 11.0, at room temperature for 1 hour with stirring. The protein is then captured on a Q-Sepharose column using the same buffer described above, and eluted with a linear gradient of 0-400 mM NaCl. The Q-Sepharose pool is then treated with 10 mM DTT, for two hours, at RT, to reduce all disulfide bonds. The pool is then diluted 10-fold so that the buffer concentration is as follows: 50 mM Tris, pH 9.0, 7 M Urea, 10 mM Cysteine, 1 mM DTT with a protein concentration of approximately 250-500 μg/ml. After another two-hour incubation under reducing conditions at RT, to obtain the protein in a free disulfide form, the pool is then dialyzed into 20 mM glycine, pH 9.0 for approximately 48 hours so that the correct disulfide bonds can be formed.

Reversed-phase HPLC chromatography, on a Vydac C18 column and 0.1% TFA/0-50% CH₃CN as a mobile phase is used as an initial purification step. This column is used to concentrate FGF-21 and removes contaminating endotoxin.

The next purification step is size exclusion chromatography on a Superdex 35/600 column performed in 1×PBS buffer, pH7.4. At this step FGF-21 is ˜95% pure. The last step involves MonoQ chromatography in 50 mM Tris, pH 8.0 and elution with a linear gradient of 0-300 mM NaCl, which usually yields >97% pure protein.

### Preparation 2

### Expression and Purification of FGF-21 in HEK293EBNA Cells

Alternatively, FGF-21 is produced in a mammalian cell expression system such as HEK293EBNA cells (EdgeBiosystems, Gaiethersburg, Md.). FGF-21 is subcloned in the proprietary expression vector representing a modification of commercially available pEAK10, between NheI and XbaI restriction sites in the MCS. The cDNA sequence encoding mature FGF-21 is fused in frame with the Igκ leader sequence to enhance secretion of the desired product in the tissue culture media. The expression is driven by the strong viral CMV promoter. HEK293EBNA cells are transiently transfected using a standard transfection reagent such as Fugene (Roche Diagnostics, Indianapolis, Ind.) and the appropriate amount of recombinant plasmid, either as a monolayer or suspension culture, at the adequate cell density. Cells are incubated at 37° C. and 5% CO₂, in serum free media, and collections are made every day for 5 days. Typically the expression level in the HEK239EBNA suspension culture is ˜30 mg/L. The expression of human FGF-21 in mammalian cells yields the natural N-terminus sequence of HPIP, i.e. without a methionine residue at the N-terminus.

### Preparation 3

### Expression and Purification of FGF-21 in Yeast

Yet another expression system for production of FGF-21 is yeast, such as Pichia pastoris, Pichia methanolica or Saccharomyces cerevisiae. For production in Pichia pastoris, a commercially available system (Invitrogen, Carlsbad, Calif.) uses vectors with the powerful AOX1 (alcohol oxidase) promoters to drive high-level expression of recombinant proteins. Alternatively, vectors that use the promoter from the GAP gene (glyceraldehyde-3-phosphate dehydrogenase) are available for high level constitutive expression. The multi-copy Pichia expression vectors allow one to obtain strains with multiple copies of the gene of interest integrated into the genome. Increasing the number of copies of the gene of interest in a recombinant Pichia strain can increase protein expression levels.

### EXAMPLE 1

### Glucose Uptake in Mouse 3T3-L1 Adipocytes

3T3-Li cells are obtained from the American Type Culture Collection (ATCC, Rockville, Md.). Cells are cultured in growth medium (GM) containing 10% calf serum in Dulbecco's modified Eagle's medium. For standard adipocyte differentiation, two days after cells reach confluency (referred as day 0), the cells are exposed to differentiation medium (DM) containing 10% fetal bovine serum, 5 μg/ml of insulin, 1 μM dexamethasone, and 0.5 μM isobutylmethylxanthine, for 48 h and then are exposed to medium containing 10% fetal bovine serum, 5 μg/ml insulin for an additional 48 h. Cells are then maintained in post differentiation medium containing 10% fetal bovine serum.

Glucose Transport Assay—FGF-21 is added to the differentiated 3T3-L1 cells in 96 well plates at 0, 0.016, 0.08, 0.4, 2, 10, or 50.0 nM, and rosiglitazone is added to a final concentration of 1 μM, Table 1. For comparison, rosiglitazone alone is added at the concentrations indicated in Table 2 or in combination with FGF-21 at 1 μg/ml. The plates are incubated at 37° C. for 72 hours.

Hexose uptake, as assayed by the accumulation of 2-deoxy-D-[¹⁴C]glucose, is measured as follows: 24 hours prior to the assay, the wells are rinsed twice with PBS and DMEM (high glucose, 1% antibiotic/antimycotic solution, 2 mM glutamine), 0.1% BSA plus FGF-21 is added. The plates are incubated at 37° C. for 72 hours. The cells are then washed twice with KRP buffer (136 mM NaCl, 4.7 mM KCl, 10 mM NaPO₄, 0.9 mM CaCl₂, 0.9 MM MgSO₄, 0.1% BSA, pH 7.4), and then KRP buffer containing 1% BSA, 2-deoxy-D-glucose, 100 μM, 0.1 μCi/well 2-deoxy-D-[¹⁴C]glucose is added and the plates are incubated at 37° C. for one hour. Cytochalasin B is added to stop further glucose uptake. Uptake is measured on a Microbeta plate reader.

The in vitro potency of FGF-21 alone or in combination with rosiglitazone is indicated in Table 1. FGF-21 alone has an ED₅₀ of 1.7 nM whereas FGF-21 in combination with rosiglitazone demonstrates a synergistic effect and has an ED₅₀ of 0.7 nM.

TABLE 1

| | FGF-21 Concentration (nM) | | | | | | |
|---|---|---|---|---|---|---|---|
| Treatment | 0.0 | .016 | .08 | 0.4 | 2.0 | 10.0 | 50.0 |
| FGF-21 | 1000* | 900 | 1000 | 1300 | 2500 | 3100 | 3500 |
| FGF-21 + | 1000 | 1500 | 2500 | 5500 | 8200 | 10000 | 11000 |
| Rosi. | | | | | | | |

*¹⁴C-Deoxyglucose (CPM)
FGF-21: 72 hr. treatment; ED₅₀ 1.7 nM
FGF-21 + Rosi.: rosiglitazone added at 1 μm, 72 hr. treatment; ED₅₀ 0.7 nM

The in vitro potency of rosiglitazone alone or in combination with FGF-2 1 at 1 μg/ml is indicated in Table 2. Rosiglitazone alone has no effect on glucose uptake in 3T3 cells whereas rosiglitazone in combination with FGF-21 has an ED₅₀ of 0.007 μM.

TABLE 2

| | Rosiglitazone Concentration (μM) | | | | | | |
|---|---|---|---|---|---|---|---|
| Treatment | 0.0 | 0.0032 | 0.016 | 0.08 | 0.4 | 2.0 | 10.0 |
| Rosi.** | 1800* | 1700 | 1800 | 1700 | 1650 | 1600 | 1600 |
| FGF-21 + | 3500 | 5000 | 8000 | 9800 | 9800 | 9800 | 10000 |
| Rosi. | | | | | | | |

*¹⁴C-Deoxyglucose (CPM)
Rosi.: rosiglitazone 72 hr. treatment
Rosi. + FGF-21: rosiglitazone + FGF-21 (1 μg/ml), 72 hr. treatment
**EC₅₀ rosiglitazone, 0.007 μM

### EXAMPLE 2

### Ob/ob Mouse Model

The Ob/ob mouse model is an animal model for hyperglycemia, insulin resistance and obesity. Male ob/ob mice are used to monitor plasma glucose levels and triglyceride levels after treatment with FGF-21, rosiglitazone, and FGF-21 in combination with rosiglitazone. The test groups of male ob/ob, mice (7 weeks old) are: FGF-21, 5 μg/day; rosiglitazone, 30 mg/kg/day; FGF-21, 3 ug/day, +rosiglitazone, 10 mg/kg/day; FGF-21, 5 ug/day, +rosiglitazone, 30 mg/kg/day; FGF-21, 5 ug/day, +rosiglitazone 10 mg/kg/day; s.c. vehicle control (0.9% NaCl, 0.1 ml/mouse); and, p.o. vehicle control (CMC/SLS/Povidone, 0.2 ml/mouse). FGF-21 is administered s.c. in 0.1 ml, and rosiglitazone is administered p.o. in 0.2 ml.

The animals are dosed daily for 14 days. Blood glucose levels are measured daily, 1 hour post dosing, using a standard protocol. The synergistic effect of FGF-21 in combination with rosiglitazone to lower plasma glucose levels as compared to FGF-21 alone is shown in Table 3.

TABLE 3

| | Blood Glucose Levels in ob/ob mice (mg/dl)* | | | | | | | |
|---|---|---|---|---|---|---|---|---|
| | Days of Treatment | | | | | | | |
| Treatment | 0 | 2 | 4 | 6 | 8 | 10 | 12 | 14 |
| Veh. Ctl. | 225 | 235 | 245 | 245 | 225 | 270 | 275 | 325 |
| (s.c.) | | | | | | | | |
| Veh. Ctl. | 225 | 230 | 170 | 190 | 135 | 190 | 160 | 300 |
| (p.o.) | | | | | | | | |
| FGF-21 | 225 | 175 | 150 | 140 | 140 | 160 | 180 | 225 |
| 5 μg/day | | | | | | | | |
| Rosi. | 225 | 125 | 150 | 135 | 115 | 125 | 120 | 115 |
| 30 mg/kg/day | | | | | | | | |
| FGF-21 | 225 | 170 | 125 | 110 | 100 | 110 | 110 | 110 |
| 3 μg/day + | | | | | | | | |
| rosi. | | | | | | | | |
| 10 mg/kg/day | | | | | | | | |
| FGF-21 | 225 | 140 | 125 | 110 | 100 | 140 | 115 | 110 |
| 5 μg/day + | | | | | | | | |
| rosi. | | | | | | | | |
| 30 mg/kg/day | | | | | | | | |
| FGF-21 | 225 | 125 | 145 | 140 | 100 | 110 | 130 | 125 |
| 5 g/day + | | | | | | | | |
| rosi. | | | | | | | | |
| 10 mg/kg/day | | | | | | | | |

*Glucose levels measured 1 hour post dose

### EXAMPLE 3

### Db/db Mouse Model

The genetically diabetic C57BL/KsJ (db/db) mouse provides an animal model of type 2 diabetes, characterized by obesity, hyperglycemia and insulin resistance with hyperinsulinemia. (Sharma et al., Am J Physiol Renal Physiol. 284(6):F1138-b 44, (2003)).

In the present study, male db/db mice, 9 weeks of age are used. The animals are randomized by body weight and blood glucose levels into groups of six mice per group. Group 1: vehicle 1: CMC/SLS/Povidone, p.o. (vehicle for rosiglitazone); Group 2: vehicle 2: 0.9% NaCl, s.c. injection (vehicle for FGF-21); Group 3: FGF-21 3 mg/day, s.c.; Group 4: rosiglitazone 10 mg/kg/day, p.o.; Group 5: FGF-21 3 mg/day, s.c.+rosiglitazone 10 mg/kg/d, p.o. The animals are dose for 14 days.

Blood glucose is measured daily, 1 hour post dosing. The combination therapy of FGF-21+rosiglitazone demonstrates a synergistic effect in lowering blood glucose levels when compared to treatment with FGF-21 or rosiglitazone alone, Table 4.

TABLE 4

| | Blood Glucose Levels in db/db mice (mg/dl)* | | | | | | | |
|---|---|---|---|---|---|---|---|---|
| | Days of Treatment | | | | | | | |
| Treatment | 0 | 2 | 4 | 6 | 8 | 10 | 12 | 14 |
| Veh. Ctl. | 265 | 305 | 300 | 300 | 325 | 335 | 360 | 370 |
| (p.o.) | | | | | | | | |
| Veh. Ctl. | 265 | 370 | 360 | 350 | 360 | 425 | 435 | 400 |
| (s.c.) | | | | | | | | |
| FGF-21 | 265 | 270 | 280 | 300 | 290 | 325 | 345 | 345 |
| 3 μg/day | | | | | | | | |
| Rosi. | 265 | 370 | 350 | 340 | 265 | 315 | 360 | 350 |
| 10 mg/kg/day | | | | | | | | |
| FGF-21 + | 265 | 250 | 240 | 215 | 205 | 200 | 190 | 190 |
| Rosi. | | | | | | | | |

*Glucose levels measured 1 hour post dose

Plasma triglycerides are measured on days 7 and 14, 1 hour post dosing. The combination therapy of FGF-21+rosiglitazone demonstrates a synergistic effect in lowering plasma triglyceride levels when compared to treatment with FGF-21 or rosiglitazone alone, Table 5.

TABLE 5

Plasma Triglycerides (mg/dL)*

| Treatment | Day 7 | Day 14 |
|---|---|---|
| Veh. Ctl. (p.o.) | 130 | 135 |
| Veh. Ctl: (s.c.) | 185 | 125 |
| FGF-21 3 ug/day | 165 | 120 |
| Rosi. 10 mg/kg/day | 135 | 85 |
| FGF-21 + Rosi.** | 85 | 65 |

*Triglyceride levels measured 1 hour post dose on days 7 and 14.
**FGF-21 + Rosi.: FGF-21 3 μg/day + Rosiglitazone 10 mg/kg/day

We claim:

1. A method for treating a mammal exhibiting type 2 diabetes or metabolic syndrome comprising: administering to said mammal a therapeutically effective amount of FGF-21 or an FGF-21 compound in combination with a thiazolidinedione sufficient to achieve in said mammal at least one of the following modifications: reduction in triglycerides, decrease in insulin resistance, reduction of hyperinsulinemia, increase in glucose tolerance, or reduction of hyperglycemia.

2. The method of claim 1 wherein said mammal is a human subject which exhibits type 2 diabetes.

3. The method of claim 1 wherein said mammal is a human subject which exhibits metabolic syndrome.

4. The method of claim 1 wherein said modification is a reduction of hyperglycemia.

5. The method of claim 1 wherein said modification is a reduction in triglycerides.

6. The method of claim 1 wherein said thiazolidinedione is selected from the group consisting of rosiglitazone or pioglitazone.

7. The method of claim 6 wherein said thiazolidinedione is rosiglitazone.

8. The method of claim 6 wherein said thiazolidinedione is pioglitazone.


SEQUENCE LISTING (2 sequences)

SEQ ID NO: 1 - protein, 181 aa, homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - DNA, 543 bp, homo sapiens
caccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcc